(19) United States
(12) Patent Application Publication
(10) Pub. No.: US 2024/0296965 A1
(43) Pub. Date: Sep. 5, 2024

(54) AVAILABILITY INDEX FOR CONNECTING WITH A USER VIA A NETWORK DEVICE

(71) Applicant: Thomson Licensing, Cesson-Sevigne (FR)

(72) Inventors: Navneeth N. KANNAN, Doylestown, PA (US); Gonzaga Joseph CHOW, Duluth, GA (US)

(21) Appl. No.: 18/116,980

(22) Filed: Mar. 3, 2023

(51) Int. Cl. G16H 80/00

(52) U.S. Cl. CPC G16H 80/00

(57) ABSTRACT

A monitoring device generates an availability index associated with the user for use by a client device associated with a remote user. The availability index is indicative of a probability of a successful connection between the remote user and the user. The availability index is based on user data associated with the user that is received from one or more sensing devices. The user data can be weighted such that user data that provides for a more accurate availability index is weight more than other user data. In this way, user data can be utilized to generate an availability index for a user that can be used by a remote user to predict a successful connection. The remote user can conserve resources as the availability index provides an accurate and reliable indicator as to the successful connection with the user.

### BACKGROUND

Network devices are increasingly being used for communication with a remote user. For example, as healthcare costs continue to increase, there is an increasing desire with users to communicate remotely with a healthcare professional, especially aging adults that desire to stay in place (in home) for extended care services or even convenience users that desire to address health issues without having to travel. While there are many individual technologies to address niche problems, given the rapid rise of connectivity technologies and the use of Artificial Intelligence techniques for medical technologies and analytical methods, these technologies can be time-consuming to actually make contact with a user, confusing, and difficult to configure making ubiquitous adoption of a particular technology unlikely. The cost of actually connecting with a remote user can be insurmountable or a deterrent to implementation of a reliable indicator as to availability of a user prior to attempting to connect with the user. Thus, there is a need for a network device that provides a reliable availability index for connecting with a user so as to provide an improved user experience.

### SUMMARY

Generally, there are many devices in the market that operate or behave as point solutions for providing medical diagnostic information associated with a health condition of a user. Each solution may have an associated device and an associated application that runs on the associated device. However, these solutions or technologies do not provide a reliable probability of the availability of a user, for example, that a user is available to receive a call, such as for diagnostic services or other service provider services. According to one or more aspects of the present disclosure a remote user, such as a trusted user, can establish a connection with a user based on an availability index associated with the user. The availability index is indicative of a probability that a connection to the user will be successful. For example, the availability index can be used by a remote user, such as a supporter or trusted user, to plan a call with a user associated with the availability index such that the remote user has a high confidence level that the user is availability and able to establish the connection via a network device.

As an example, in remote care settings, such as telehealth, professional caregiver, aging-in-place, or personal home care, remote users (also referred to as supporters or trusted users), such as physicians, nurses, other medical professionals, other caregivers, family members, etc., can expend resources simply attempting to make a connection with the individual seeking services (a user). Such remote users do not know when the user is available to receive a connection, for example, to establish a communication, such as video conference. For example, the user can be elsewhere, not present at the site or within proximity to a particular device such as a video conference device, sleeping or not within proximity of a monitoring device, and/or avoiding calls or other contact due to spam or unwanted solicitations. Often, this results in many unsuccessful attempts to make a connection with the user. The unavailability of the user may also result in uncertainty as to the care of the user. While a schedule can be useful, a schedule itself has uncertainty given the lack of adherence or interruptions that naturally are associated with a schedule. The present disclosure provides a multi-modal approach to understanding the behavior of a user so as to provide improved service through successful connections between the user and the remote user based on user data collected or received from various sensing devices associated with the user so as to generate an availability index that can be utilized by the remote user to strategically make connection attempts with the user. Such connection attempts based on an availability index have more probability of a success and thus conserve valuable resources for both the remote user and the user.

One or more novel solutions of the present disclosure provide for generating an availability index associated with a user of a network device based on user data, one or more correlating factors, or both. User data can be received from a network device that receives data from one or more sensors associated with the user. The one or more sensors can be part of or included within the network device or wired and/or wirelessly connected or otherwise coupled to the network device. The user data can be associated with one or more correlating factors that each have one or more correlating factor values associated with a correlating factor index. The availability index is updated based on information, such as the user data, one or more correlating factors, or both so that the probability that a user can make a connection is reliable. In this way, a remote user can optimize one or more services provided to the user. For example, the remote user will not incur loss of time or costs associated with unsuccessful attempts to connect with a user. Rather, the remote user has a way to measure the success of a connection with the user and thus can appropriately plan or be alerted to best opportunities (high probability) to connect with a user.

An aspect of the present disclosure provides a network device for generating an availability index associated with a user. The network device comprises a memory storing one or more computer-readable instructions and a processor configured to execute the one or more computer-readable instructions to receive user data associated with the user from one or more sensing devices, generate the availability index based on the user data and one or more correlating factors, receive a request for the availability index from a client device associated with a remote user, and send the availability index to the client device.

In an aspect of the present disclosure, the processor is further configured to execute the one or more instructions to apply a factor index associated with the one or more correlating factors to the user data, wherein the availability index is generated based on applying the factor index to the user data.

In an aspect of the present disclosure, the processor is further configured to execute the one or more instructions to assign the secondary multi-sensor device to receive a request from the client device to initiate a connection with the client device, and establish the connection with the client device.

In an aspect of the present disclosure, the processor is further configured to execute the one or more instructions to store the availability index at a network resource accessible by a service portal system.

In an aspect of the present disclosure, the availability index is based on a profile configuration associated with the user.

In an aspect of the present disclosure, the network device is a monitoring device that comprises at least one of the one or more sensing devices.

In an aspect of the present disclosure, the processor is further configured to execute the one or more instructions to alter one or more factor indexes applied to the user data based on historical user data and one or more associated successful connections.

An aspect of the present disclosure provides a method for a network device to generate an availability index associated with a user. The method comprises receiving user data associated with the user from one or more sensing devices, generating the availability index based on the user data and one or more correlating factors, generating the availability index based on the user data and one or more correlating factors, and sending the availability index to the client device.

In an aspect of the present disclosure, the method further comprises applying a factor index associated with the one or more correlating factors to the user data, wherein the availability index is generated based on applying the factor index to the user data.

In an aspect of the present disclosure, the method further comprises receiving a request from the client device to initiate a connection with the client device, and establishing the connection with the client device.

In an aspect of the present disclosure, the method further comprises storing the availability index at a network resource accessible by a service portal system.

In an aspect of the present disclosure, the method such that the availability index is based on a profile configuration associated with the user.

In an aspect of the present disclosure, the method such that the network device is a monitoring device that comprises at least one of the one or more sensing devices.

In an aspect of the present disclosure, the method further comprises altering one or more factor indexes applied to the user data based on historical user data and one or more associated successful connections.

An aspect of the present disclosure provides a non-transitory computer-readable medium of a network device storing one or more instructions for generating an availability index associated with a user, which when executed by a processor of the network device, cause the network device to perform one or more operations including the steps of the methods described above.

According to various aspects of the present disclosure, one or more novel solutions can provide a reliable availability index associated with a user of a network device so as to provide an improved quality of service and reduce costs associated with a remote user connecting with the user via the network device.

### BRIEF DESCRIPTION OF DRAWINGS

In the drawings, like reference numbers generally indicate identical, functionally similar, and/or structurally similar elements.

FIG. 1 is a schematic diagram of a network environment, according to one or more aspects of the present disclosure;

FIG. 2 is a more detailed block diagram illustrating various components of a network device, according to one or more aspects of the present disclosure;

FIG. 3 is an illustration of a monitoring device in a network environment, according to one or more aspects of the present disclosure;

FIG. 4 is an illustration of a connection between a user and a remote user, according to one or more aspects of the present disclosure;

FIG. 5 illustrates a process for a monitoring device to generate an availability index associated with a user, according to one or more aspects of the present disclosure;

FIG. 6 illustrates a table for one or more correlating factors, according to one or more aspects of the present disclosure; and

FIG. 7 is a flow chart for generating an availability index associated with a user, according to one or more aspects of the present disclosure.

### DETAILED DESCRIPTION

The following detailed description is made with reference to the accompanying drawings and is provided to assist in a comprehensive understanding of various example embodiments of the present disclosure. The following description includes various details to assist in that understanding, but these are to be regarded merely as examples and not for the purpose of limiting the present disclosure as defined by the appended claims and their equivalents. The words and phrases used in the following description are merely used to enable a clear and consistent understanding of the present disclosure. In addition, descriptions of well-known structures, functions, and configurations may have been omitted for clarity and conciseness. Those of ordinary skill in the art will recognize that various changes and modifications of the examples described herein can be made without departing from the spirit and scope of the present disclosure.

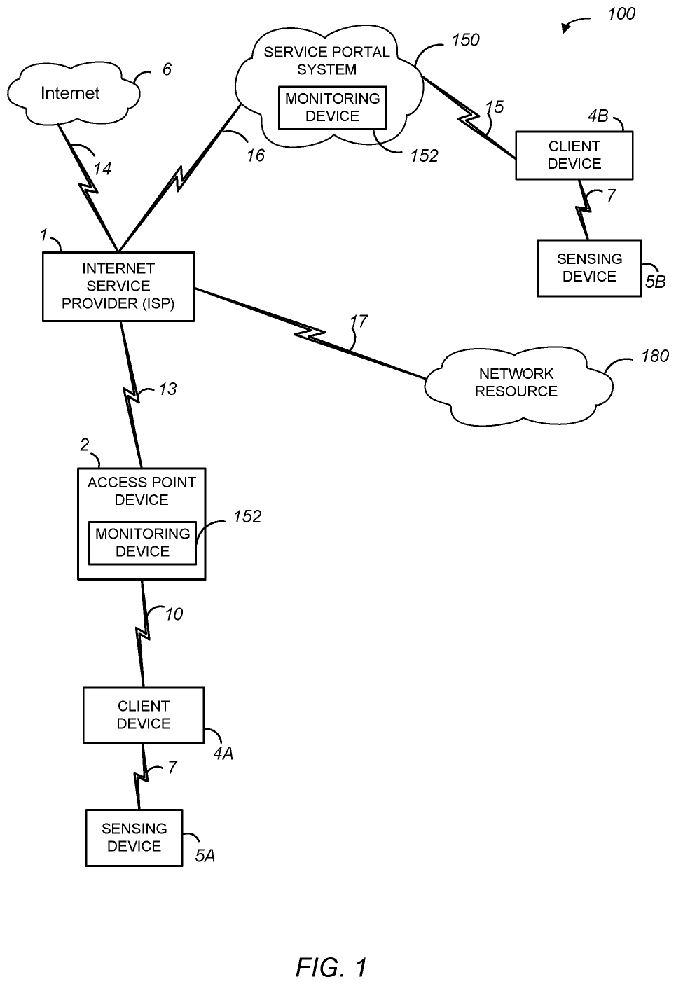

FIG. 1 is a schematic diagram of a network environment 100, according to one or more aspects of the present disclosure. For example, a network environment can provide for the accurate generation of an availability index associated with a user. An example network environment can be related to a diagnostic network, a caregiving network, and/or any other network for providing one or more services for a user (such as a patient) such that one or more aspects associated with the user (for example, biometric data (such as from a third-party user-wearable device), an audio and/or visual interface, etc.) can be utilized to provide accurate probability of success of a connection to a user by a remote user based on an availability index.

It should be appreciated that various example embodiments of inventive concepts disclosed herein are not limited to specific numbers or combinations of devices, and there may be one or multiple of some of the aforementioned electronic apparatuses in the network environment, which may itself consist of multiple communication networks and various known or future developed wireless connectivity technologies, protocols, devices, and the like.

As shown in FIG. 1, the main elements of the network environment 100 include a network comprising an access point device 2 connected to any of the Internet 6, a service portal system 150, a network resource 180, any other cloud storage and/or repository, or any combination thereof via an Internet Service Provider (ISP) 1 and also connected to different wireless devices or network devices such as one or more client devices 4A and 4B (collectively referred to as client device(s) 4), and one or more sensing devices 5A and 5B (collectively referred to as sensing device(s) 5). The network environment 100 shown in FIG. 1 includes wireless network devices (for example, one or more client devices 4) that may be connected in one or more wireless networks within the network environment 100. Additionally, there could be some overlap between wireless devices in the different networks. That is, one or more network or wireless devices could be located in more than one network.

The ISP 1 can be, for example, a content provider or any computer for connecting the access point device 2 to any of a network resource 180, the Internet 6, the service portal system 150, or any combination thereof via the ISP 1. For example, Internet 6 can be a cloud-based service that provides access to a network resource 180, such as a cloud-based repository, accessible via ISP 1 where the network resource 180 comprises information associated with or an access requested by any one or more network devices of the network environment 100. The network resource 180 can comprise any of a website, a database, a portal, any other repository for storing and retrieving information, or any combination thereof, for example, information associated with a user (such as a patient) for connection with a remote user (such as a support and/or trusted user, for example, any of a healthcare services provider, a nurse, a doctor, a caregiver, a family member (such as a primary contact), a coordinator a personal services, an authorized consent provider, any other healthcare professional or other provider, or any combination thereof).

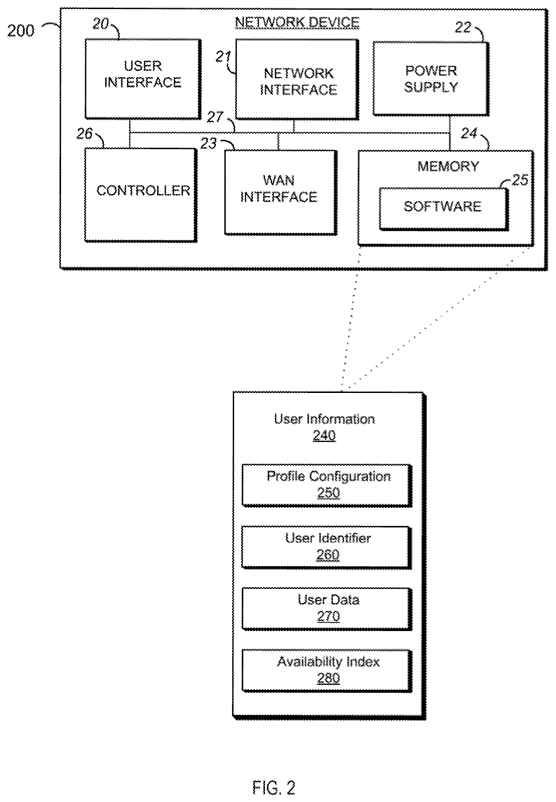

The service portal system 150 can provide monitoring, aggregation and/or controlling of data associated with a user that is associated with one or more sensing devices 5, a query or both in the network environment 100. For example, the service portal system 150 can comprise one or more databases or repositories hosted in the cloud and/or locally. The one or more databases can store user information, such as user information 240 discussed with reference to FIG. 2, associated with a user, such as data related to caregiving for the user. For example, the service portal system 150 can comprise a monitoring device 152 or be coupled to a monitoring device 152. The user information 240 can be collected by and/or received from one or more sensing devices 5 at the monitoring device 152 and sent by the monitoring device 152 to one or more network devices of the network environment 100, such as service portal system 150 and/or any one or more other network devices. The user information can comprise any data associated with the user. The service portal system 150 can aggregate user information from one or more queries associated with one or more users and can apply an algorithm, such as an artificial intelligence algorithm and/or a machine learning technique, to analyze a pattern or trend associated with aggregated results so as to generate an availability index associated with the user that indicates an availability of a user to communicated with a remote user. In one or more embodiments, the service portal system 150 can communicate with any one or more external repositories of Internet 6 via ISP 1 or internal repositories, such as a notification repository. In one or more embodiments, any of the sensing devices 5 can be directly or indirectly coupled to the service portal system 150 and/or one or more other network devices, such as any of an access point device 2, a client device 4, a monitoring system 152, any other network devices, or any combination thereof. The connection 14 between the Internet 6 and the ISP 1, the connection 17 between the ISP 1 and the network resource 180, the connection 16 between the service portal system 150 and the ISP 1, the connection 15 between the service portal system 150, the client device 4B, and the connection 13 between the ISP 1 and the access point device 2, and any other connection 10 and/or connection 7 can be implemented using a wide area network (WAN), a virtual private network (VPN), metropolitan area networks (MANs), system area networks (SANs), a data over cable service interface specification (DOCSIS) network, a fiber optics network (for example, FTTH (fiber to the home) or FTTX (fiber to the x), or hybrid fiber-coaxial (HFC)), a digital subscriber line (DSL), a public switched data network (PSDN), a global Telex network, or a 2G, 3G, 4G, 5G, or 6G network, for example. Any of the connections 7, 10, 13, 14, 15, 16, and 17 or any combination thereof (collectively referred to as network connections or connections) can further include as some portion thereof a broadband mobile phone network connection, an optical network connection, or other similar connections.

The access point device 2 can be, for example, an access point and/or a hardware electronic device that may be a combination modem and gateway that combines the functions of a modem, an access point (AP), and/or a router for providing content received from the ISP 1 to one or more network devices (for example, one or more client devices 4) in the network environment 100, or any combination thereof. In one or more embodiments, the access point device 2 can comprise a monitoring device 152 as discussed below. It is also contemplated by the present disclosure that the access point device 2 can include the function of, but is not limited to, a universal plug and play (UPnP) simple network management protocol (SNMP), an Internet Protocol/Quadrature Amplitude Modulator (IP/QAM) set-top box (STB) or smart media device (SMD) that is capable of decoding audio/video content, and playing over-the-top (OTT) or multiple system operator (MSO) provided content. The access point device 2 may also be referred to as a residential gateway, a home network gateway, or a wireless access point (AP). The access point device 2 can comprise and/or be coupled to a monitoring device 152 so as to generate an availability index associated with a user based on user information, such as user data, analyzed by the monitoring device 152. The present disclosure contemplates that the monitoring device 152 is coupled to the access point device 2 and/or a service portal system 150 and a client device 4 and/or a sensing device 5.

The one or more client devices 4 can be, for example, hand-held computing devices, personal computers, electronic tablets, mobile phones, smart phones, smart speakers, Internet-of-Things (IoT) devices, iControl devices, portable music players with smart capabilities capable of connecting to the Internet, cellular networks, and interconnecting with other devices via Wi-Fi and Bluetooth, or other wireless hand-held consumer electronic devices capable of executing and displaying content received through the access point device 2. Additionally, the client devices 4 can be a television (TV), an IP/QAM set-top box (STB) or a streaming media decoder (SMD) that is capable of decoding audio/video content, and playing over OTT or MSO provided content received through the access point device 2. In one or more embodiments, the client devices 4 can comprise any network device associated with a user for interacting with any type of one or more sensing devices 5. For example, the client device 4 can interact with a plurality of sensing devices 5 such that each sensing device 5 senses user data associated with a user or an environment, such as any of biometric data or information, environmental data, motion, any other data associated with a user, or any combination thereof. In one or more embodiments, one or more sensing devices 5 are included within or local to (built-in) the client device 4. In one or more embodiments, the client device 4 can be a network device that includes a monitoring system 152 or is part of a service portal system 150.

One or more sensing devices 5 can connect to one or more client devices 4, for example, via a connection 7. The present disclosure contemplates that any one or more sensing devices can be coupled directly or indirectly to a monitoring device 152. The one or more sensing devices 5 can comprise one or more devices for generating, sending, and/or receiving user information, for example user data, such as any of one or more diagnostic devices (for example, any of an optical instrument (such as a camera, an image capture device, or any other visual user interface device, any device for capturing an image, a video, a multi-media video, a pulse oximeter or any other oxygen saturation sensing system, a blood pressure monitor, a temperature sensor, a glucose monitor, any other type of data device, a biometric sensor, a biometric tracker or sensor, ambient temperature sensor (such as a thermometer), or any combination thereof), a light sensor, a humidity sensor, a motion detector (such as, an infrared motion sensor or Wi-Fi motion sensor), a facial recognition system, a voice recognition system, an input and/or output device (I/O device), such as any of a receiver (such as, a microphone (for example, a far field voice (FFV) microphone) and/or other voice capture system), a transmitter (such as a speaker), or both, any other sensing device, or any combination thereof.

It is contemplated by the present disclosure that the network devices, such as any of the service portal system 150, a monitoring device 152, the access point device 2, the one or more client devices 4, the one or more sensing device 5, or any combination thereof include electronic components or electronic computing devices operable to receive, transmit, process, store, and/or manage data and information associated with the network environment 100, which encompasses any suitable processing device adapted to perform computing tasks consistent with the execution of computer-readable instructions stored in a memory or a computer-readable recording medium (for example, a non-transitory computer-readable medium). Further, any, all, or some of the computing components in the network devices may be adapted to execute any operating system, including Linux, UNIX, Windows, MacOS, DOS, and ChromOS as well as virtual machines adapted to virtualize execution of a particular operating system, including customized and proprietary operating systems. The network devices are further equipped with components to facilitate communication with other computing devices or other network devices over the one or more network connections to local and wide area networks, wireless and wired networks, public and private networks, and any other communication network enabling communication in the network environment 100. Any one or more network devices of network environment 100 can comprise a network device 200 as illustrated in FIG. 2.

FIG. 2 is a more detailed block diagram illustrating various components of an exemplary network device 200, such as a network device comprising any of a service portal system 180, a monitoring device 152, an access point device 2, a client device 4, a sensing device 5, any other network device, or any combination thereof implemented in the network environment 100 of FIG. 1, according to one or more aspects of the present disclosure. The network device 200 can be, for example, a computer, a server, any other computer device with smart capabilities capable of connecting to the Internet, cellular networks, and interconnecting with other network devices via Wi-Fi and Bluetooth, or other wireless hand-held consumer electronic device capable of providing management and control of data, for example, a monitoring system 180, according to one or more aspects of the present disclosure. The network device 200 includes one or more internal components, such as a user interface 20, a network interface 21, a power supply 22, a controller 26, a WAN interface 23, a memory 34, and a bus 27 interconnecting the one or more elements.

The power supply 22 supplies power to the one or more internal components of the network device 200 through the internal bus 27. The power supply 22 can be a self-contained power source such as a battery pack with an interface to be powered through an electrical charger connected to an outlet (for example, either directly or by way of another device). The power supply 22 can also include a rechargeable battery that can be detached allowing for replacement such as a nickel-cadmium (NiCd), nickel metal hydride (NiMH), a lithium-ion (Li-ion), or a lithium Polymer (Li-pol) battery.

The user interface 20 includes, but is not limited to, push buttons, a keyboard, a keypad, a liquid crystal display (LCD), a thin film transistor (TFT), a light-emitting diode (LED), a high definition (HD) or other similar display device including a display device having touch screen capabilities so as to allow interaction between a user and the network device 200, for example, for a user to enter any one or more profile configurations 250, a user identifier 260, any other information associated with a user or network device, or a combination thereof that are stored in memory 24. The network interface 20 can include, but is not limited to, various network cards, interfaces, and circuitry implemented in software and/or hardware to enable communications with and/or between the monitoring system 180, the access point device 2, an extender access point device 3, and/or a client device 4 using any one or more of the communication protocols in accordance with any one or more connections (for example, as described with reference to FIG. 1). In one or more embodiments, the user interface 20 and/or the network interface 21 enables communications with a sensing device 5, directly or indirectly.

The memory 24 includes a single memory or one or more memories or memory locations that include, but are not limited to, a random access memory (RAM), a dynamic random access memory (DRAM) a memory buffer, a hard drive, a database, an erasable programmable read only memory (EPROM), an electrically erasable programmable read only memory (EEPROM), a read only memory (ROM), a flash memory, logic blocks of a field programmable gate array (FPGA), an optical storage system, a hard disk or any other various layers of memory hierarchy. The memory 24 can be used to store any type of instructions, software, or algorithms including software 25, for example, a monitoring application for controlling the general function and operations of the network device 200 in accordance with one or more embodiments. In one or more embodiments, memory 24 can store user information 240, such as any of one or more profile configurations 250 associated with one or more user identifiers 260, user data 270, for example, received from one or more sensing devices 5 and/or other network devices, availability index 280, or any combination thereof. In one or more embodiments, any of the user information 240 can be stored locally at the network device 200, such as in memory 24, or remotely, such as at a network resource 180, a service portal system 150, or both. The one or more user identifiers 260 can comprise a unique identifier associated with one or more users, one or more network devices, or both so as to uniquely identify a profile configuration 250. The one or more user identifiers 260 can be associated with one or more profile configurations 250 which include information associated with one or more profiles of one or more users. The network device 200, such as a service portal system 150, can manage and control access to data associated with the one or more user identifiers 260 based on the one or more profile configurations 250. For example, the service portal system 150 can send a notification to a remote user associated with a user based on a profile configuration 250 associated with a user, such as a client user associated with a user identifier 260. The user data 270 can comprise any data associated with a user, such as data received from one or more sources, for example, a sensing device 5, connected or coupled to or within the network device 200. The availability index 280 can be determined based on any of the user data 270, any other user information 240, any other data, or any combination thereof. The availability index 280 is indicative of a probability of a connection to the associated user being successful. For example, a high availability index 280 (a large value, such as 10) can be indicative of a high probability of success of a remote user connecting with the user and a low availability index 280 (a low value, such as 1) can be indicative of a low probability of success of a remote user connecting with the user. The availability index 280 can be based on any system or range, for example, a range of 1-10 with the endpoints associated with either a highest availability index 280 or a lowest availability index 280.

The controller 26 controls the general operations of the network device 200 and includes, but is not limited to, a central processing unit (CPU), a hardware microprocessor, a hardware processor, a multi-core processor, a single core processor, a field programmable gate array (FPGA), a microcontroller, an application specific integrated circuit (ASIC), a digital signal processor (DSP), or other similar processing device capable of executing any type of instructions, algorithms, or software including the software 25 which can include a monitoring application in accordance with one or more embodiments. Communication between the components (for example, 20-26) of the network device 200 may be established using an internal bus 27.

The network interface 21 can include various network cards, interfaces, and circuitry implemented in software and/or hardware to enable communications with any one or more other network devices, for example, any of a client device 4, ISP 1, any other network device (for example, as described with reference to FIG. 1), or a combination thereof. The communications can utilize a visual interface connection that allows for a visual interface between a user and a remote user, for example, a communication that utilizes an optical instrument (such as for a video call or for an image capture). For example, the network interface 21 can include multiple radios or sets of radios (for example, a 2.4 GHz radio, one or more 5 GHz radios, and/or a 6 GHz radio), which may also be referred to as wireless local area network (WLAN) interfaces. In one or more embodiments, one radio or set of radios (for example, 5 GHz and/or 6 GHz radio(s)) provides a backhaul connection between the wireless extender access point device 3 and the access point device 2. In one or more embodiments, the monitoring system 180 is connected to or is part of the access point device 2 such that a backhaul connection is established between the monitoring system 180 and one or more wireless extender access point devices 3. Another radio or set of radios (for example, 2.4 GHz, 5 GHZ, and/or 6 GHz radio(s)) provides a fronthaul connection between the extender access point device 3 and one or more client device(s) 4.

The wide area network (WAN) interface 23 may include various network cards, and circuitry implemented in software and/or hardware to enable communications between the access point device 2 and the ISP 1 using the wired and/or wireless protocols in accordance with connection 13 (for example, as described with reference to FIG. 1).

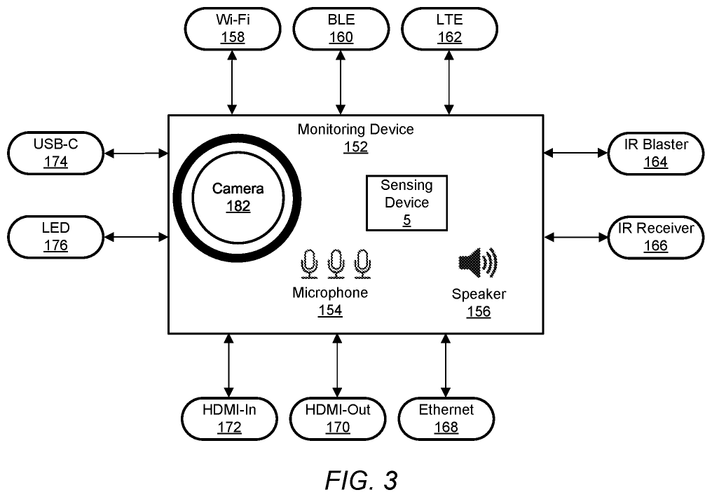

FIG. 3 illustrates a monitoring device 152, according to one or more aspects of the present disclosure. The monitoring device 152 can comprise a network device 200 as discussed with reference to FIG. 2. Monitoring device 152 can comprise any of one or more I/O devices, such as any of an optical instrument or image capture device (such as a camera 182 or any other device that can obtain one or more visuals of a user), an audio input device (such as a microphone 154, a microphone array, a far field voice (FFV) solution, a voice assistant, etc.), an audio output device (such as a speaker 156), any other I/O device, or any combination thereof, a sensing device 5, any other source of user data, or any combination thereof. In one or more embodiments, a service portal system 150 is connected directly and/or indirectly to the monitoring device 152. In one or more embodiments, any one or more components of the monitoring device 152 can be included within or connected to the monitoring device 152. The monitoring device 152 can include any of one or more ports or receivers, for example, a Wi-Fi (such as a Wi-Fi5 (dual-band simultaneous (DBS)) port 158, a BLE port 160, an LTE port 162, an infrared (IR) blaster port 164, and IR receiver port (166), an Ethernet port 168, an HDMI-Out port 170, an HDMI-In port 172, an external power supply (such as a universal serial bus type-C(USB-C)), an LED output 176, or any combination thereof. The sensing device 5 can include any one or more types of sensors associated with any type of measurement, such as any of a power sensor, a temperature sensor, a light sensor, a humidity sensor, a motion sensor, a biometric sensor (such as a blood pressure monitor, oxygen saturation meter, pulse meter, etc.), any other type of sensor, or any combination thereof. In one or more embodiments, the sensing device 5 can be an IoT device.

The monitoring device 152 can utilize one or more artificial intelligence technologies, one or more algorithms, and/or one or more functions to analyze data associated with a user (such as user data 270) so as to generate an availability index 280 associated with the user for use by a remote user to establish a connection with the user. The monitoring device 152 can receive data associated with the user (such as user data 270) from any of one or more sensor devices 5 (such as any of a biometric device, a health diagnostic device, an environmental device, any other sensing device associated with a user, or any combination thereof), an optical instrument, an audio input device, the user interface, such as a keyboard, a mouse, a remote control, a touchscreen, a BLE device, any other user interface device, or any combination thereof), any other input, or any combination thereof. As an example, the monitoring device 152 can transmit a request to the user via an audio output device and receive a response as data via any of the audio input device, the camera, any one or more sensor devices 5, or any combination thereof. In addition, the monitoring device 152 can retrieve data associated with the user from a network or cloud resource, such as from the Internet 6, a repository (for example, a service portal system 150), or both. All the data received can be aggregated and analyzed by the monitoring device 152, the service portal system 150 or both to provide a remote user or remote network device with an availability index 280 associated with the user. The monitoring device 152 can indicate to the remote user, for example, that the user associated with the monitoring device 152 is available to connect, such as via a video conference. The monitoring device 152 can access user information 240 associated with a user so as to generate the availability index 280. In one or more embodiments, the service portal system 150, the monitoring device 152, or both can utilize the user data 270 to generate an availability index so as to predict availability of the user for a particular type of connection, such as a video conference, a telephone call, a messaging interface, a text-based interface, any other connection, or any combination thereof with a remote user.

The monitoring device 152 can be connected to and/or include any of one or more sensing devices 5, one or more client devices 4, any other device, or any combination thereof. For example, for in-place diagnosis, the monitoring device 152 can collect data from a variety of sources, such as one or more sensing devices 5, with different capabilities or functions. In addition to functional differences, the one or more sensing devices 5 can have differences in the protocol or method of communication of data, including user data 270 sent to and/or received by the monitoring device 152.

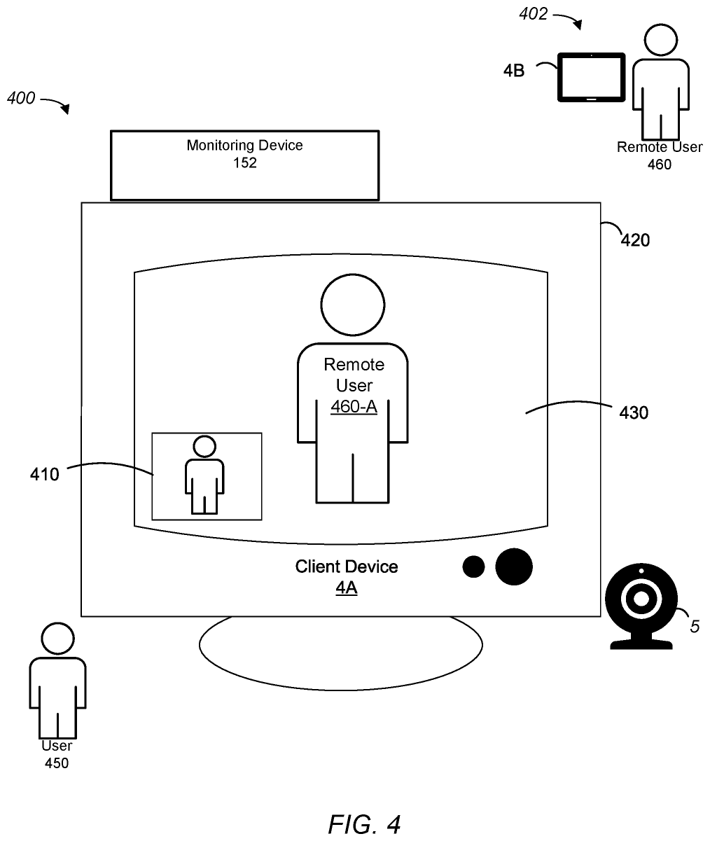

FIG. 4 is an illustration of is an illustration of a connection between a user 450 of a network environment 400 associated with a monitoring device 152 and a client device 4a and a remote user 460 of a network environment 402 associated with a client device 4B, according to one or more aspects of the present disclosure. The network environment 402 can be remote from the network environment 400. Either or both of the network environments 402 and/or 400 can be similar to or the same as the network environment 100 of FIG. 1. A monitoring device 152 can be a network device 200 that is connected to an output device, such as a client device 4B, that includes a user interface 20, such as a display device 420 used for a connection between the remote user 460 and the user 450. In one or more embodiments, the monitoring device 152 is connected to a sensing device 5 or comprises a sensing device 5 that comprises an interactive interface, such as a camera or a visual interface.

A user 450 in a network environment 400 can be within a proximity of the monitoring device 152 such that the sensing device 5, senses the user 450. For example, sensing device 5 can comprise a motion sensor, an I/O device (such as a camera, a microphone, or both), and/or any other device capable of detecting and/or recognizing user 450. The sensing device 5 sends user data 270 to the monitoring device 152. The monitoring device 152 can generate an availability index 280 based on the user data 270 and/or any other data, such as any of previously received user data 270, a profile configuration 250 associated with the user 450 (such as information that indicates a schedule of the user 450), any other information associated with the user, or any combination thereof. The monitoring device 152 can send the client device 4B the availability index 280. The client device 4B can provide the availability index 280 to the remote user 460, for example, via any of an alert (such as a sound, a visual indicator, or both), a message, an electronic mail, any other electronic correspondence, =an indicator (such as a light emitting diode (LED), any other visual indicator including, but not limited to, an indicator of a graphical user interface, or any combination thereof), a voice message, a telephone call, or any combination thereof. The remote user 460 can establish a connection with the user 450 based on the availability index 280. For example, the client device 4B can determine that the probability indicated by the availability index 280 that a connection to the user would be successful based on a comparison of the availability index 280 to a threshold. The client device 4B can, based on the comparison, establish a connection with any of the client device 4A, the monitoring device 152, any other network device 200 associated with the client user 450 (such as a cellular telephone), or any combination thereof.

In the example illustrated in FIG. 4, the remote user 460 and the user 450 are connected via the client device 4A and the client device 4B via, for example, a teleconferencing application. The remote user 460 is visually available to the user 450 via a screen 430 (as an image 460-A of the remote user 460) of the display device 420 with the reflection of the user 450 available in a picture-in-picture window 410 of the screen 430. In one or more embodiments, the client device 4A is part of or included within the monitoring device 152.

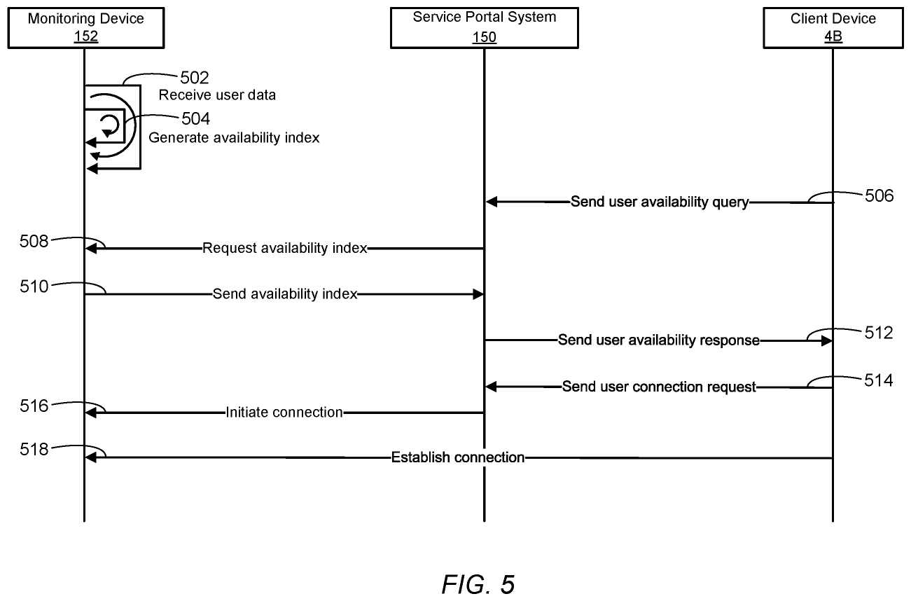

FIG. 5 illustrates a process for a monitoring device 152 to generate an availability index 280 associated with a user, according to one or more aspects of the present disclosure. A monitoring device 152 can comprise or be a network device 200 that is connected to or comprises one or more sensing devices 5. The monitoring device 152 can be associated with a user within a network environment. The monitoring device 152 can receive at 502 user data associated with the user from one or more sensing devices 5. The user data 270 can be indicative of any of a location of the user, such as whether the user is within proximity of the monitoring device 152, a state of the user, such as any of awake, active, resting, asleep, exercising, any other state, or any combination thereof. For example, the user data can comprise any type of data from one or more sensing devices 5, such as any of an I/O device data (such as any of audio data, visual data, multi-media data, a voice recognition system, a facial recognition system, any other data from one or more I/O devices, or any combination thereof), biometric data (such as data from a biometric device including, but not limited to, any of pulse oximeter, a blood pressure monitor, a temperature sensor, a glucose monitor, a tracker, any other biometric device, or any combination thereof), one or more environmental parameters (such as data from any of a light sensor, a noise sensor, a motion detector, a humidity sensor, a temperature sensor, any other environmental measurement device, or any combination thereof), any other data associated with a user, or any combination thereof.

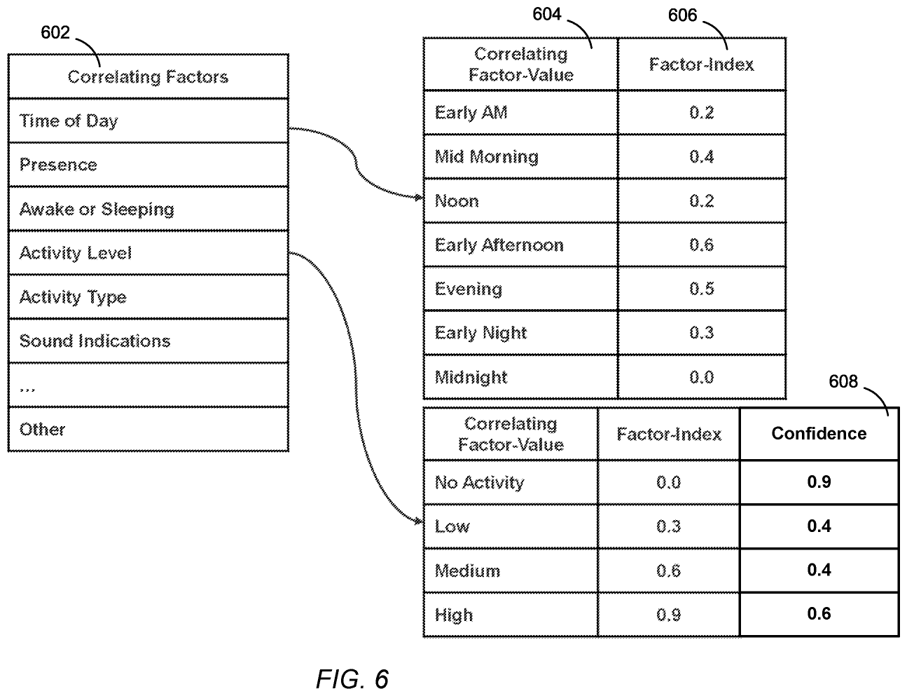

The monitoring device 152 can generate an availability index 280 at 504. The availability index 280 is indicative of the success of a connection to a user. For example, the monitoring device 152 can generate the availability index 280 based on any of the user data 270, one or more weights associated with the user data 270, a profile configuration 250, any other user information 240, a schedule associated with the user, historical information associated with previous attempts to establish a communication with the user (a successful connection, an unsuccessful connection, or both), or any combination thereof. A weight can be a factor index 606 as discussed with reference to FIG. 6. The weight can be modified by a confidence 608 as discussed with reference to FIG. 6. As an example, a successful connection (for example, an answered call) can be used to alter or modify one or more weights dynamically, such as to increase a weight, whereas an unsuccessful connection (for example, an unanswered call) can be used to alter or modify one or more weights, such as to decrease a weight. The monitoring device 152 can receive user data 270 directly associated with the user (such as a state of the user, biometric data, voice and/or facial recognition, etc.) and/or indirectly associated with the user (such as one or more signals from a remote control device associated with a client device 4 of the user (for example, data that indicates a television is displaying content, light sensor data indicating that a light source is active in a particular location, sound data indicating movement by the user (for example, sounds of cooking, cleaning, sleeping, laughter, etc.), any other indirectly associated data, or any combination thereof).

The monitoring device 152 can receive user data 270 at any timing frequency, such as any of periodically, in real-time, upon request (for example, a request from one or more network devices 200 (such as a service portal system 150, a client device 4 associated with a remote user, or both) a user input, or both), at timed intervals, based on a semaphore, continuously, at completion of an interrupt and/or timer, any other time, or any combination thereof. The monitoring device 152 can generate the availability index 280 any of at each instance of receiving user data 270, after aggregation of the user data 270, after applying a formula or algorithm to the user data 270 (such as any of averaging for a period of time, determining a median, after applying any function, artificial intelligence (AI) process, or any other processing, or any combination thereof), any other analysis, or any combination thereof. For example, the availability index 280 can be based on user data 270 and/or analyzed or processed user data 270. Prior to generating an availability index 280, the monitoring device 152 may determine that a certain amount of data has been collected in a time period, for example, so that outlier user data can be excluded from the analysis of the availability index 280.

In one or more embodiments, the availability index 280 can be based on any of the user data 270 in combination with historical user data 270, a profile configuration 250, any other user information, or any combination thereof. For example, a schedule associated with a profile configuration 250 along with user data 270 can be processed to determine an availability index 280. As an example, if a schedule indicates that at a given time the user is asleep and the user data 270 indicates a sound has been detected, the availability index 280 can be set to a low value as the sound is likely not from the user such that an attempt to make a connection with the user has a low probability of success. As another example, if the schedule indicates that at a given time the user is exercising, the availability index 280 can be set to a middle value as the user is busy even though the user data 270 indicates the user is awake and active such that an attempt to make a connection with the user has a mid range or average probability of success. As another example, if the schedule indicates that at a given time the user is free and the user data 270 indicates that the user is awake, active or within proximity of the monitoring device 152, the availability index can be set to a high value such that an attempt to make a connection with the user has a high probability of success.

In one or more embodiments, the availability index 280 is based on one or more weights applied to data used for generation of the availability index 280. For example, a weight can be assigned to user data based on one or more factors, such as any of a type of sensing device 5 (for example, any of a biometric device, an I/O device, an environmental measurement device, any other sensing device, a remote control, an access point device, or any combination thereof), a time (for example, any of time of day, day of week, day of month, month of year, year, any other timing trigger, or any combination thereof), a frequency (for example, how often a measurement from a sensing device is received, whether the measurement is in real-time or delayed, or both), a type of monitoring (for example, active monitoring (such as biometric data), passive monitoring (such as monitoring of a remote control), any other factor, or any combination thereof. For example, more weight may be given to user data associated with a time of day associated with wake hours and/or an active monitoring while less weight may be given to user data associated with a time of day associated with sleep hours and/or a passive monitoring. In this way, user data can be weighted so as to give user data that has been historically associated with a successful connection to the user can be given more weight than other user data.

A client device 4B associated with a remote user, such as any of a caregiver, a healthcare professional, a service provider, a family member, any other remote user associated with a profile configuration 250 associated with the user that indicates that the remote user has permission to connect with the user, any other remote user that has been previously authorized, any other remote user that has been authenticated (for example, using any of a password, username, quick response (QR) code, one time passcode (OTP), or any combination thereof), or otherwise trusted user, or any combination thereof, can send to a service portal system 150 a user availability query at 506. The user availability query can comprise a request for the user availability index 280 associated with a user.

The service portal system 150 can receive the user availability query at 506. The service portal system 150 can query a network resource 180 for the user availability index 280 and provide such to the client device 4B, for example, if the user availability index 280 is not stale (for example, an update to the user availability index 280 occurred within an update time threshold), can request the availability index 280 to the monitoring device 152 at 508, or both. The monitoring device 152 can receive the request for the availability index 280 at 508 and can send a response at 510 to the service portal system 150. The response can comprise any of the availability index 208 that is generated at the time of the request at 508, previously at 504, or any other time, other information (such as a user identifier 260), any other user information 240 and/or other data, or any combination thereof.

The availability index 280 can be generated based on one or more correlating factors and user data. For example, FIG. 6 illustrates a table for one or more correlating factors 602. The one or more correlating factors can 602 indicate a weight associated with user data. For example, the one or more correlating factors 602 can comprise any of a time of day factor, a presence factor, an awake or sleeping factor, an activity level factor, an activity type factor, a sound indications factor, any other factor, or any combination thereof. Each of the one or more correlating factors 602 can have one or more associated correlating factor values 604. For example, the correlating factor 602 of time of day can be associated with one or more correlating factor values 604 of any of early AM, mid morning, noon, early afternoon, evening, early night, midnight, any other time of day, or any combination thereof and the correlating factor 602 of activity level can be associated with one or more correlating factor values 604 of any of no activity, low, medium, high, any other activity level indication, or any combination thereof. Each of the correlating factor values 604 can be associated with a factor index 606 (also referred to as a weight), a confidence 608, or both, for example, as shown in FIG. 6. The factor index 606 can indicate a probability of a correlating factor-value 604. For example, a low factor index 606 (such as 0.0 or 0.2) can indicate a low probability while a higher factor index 606 can indicate a high probability (such as 0.7 or 0.9). A correlating factor value 604 can also be associated with a confidence 608. The confidence 608 indicates the reliability of the weight or factor index 606. For example, a confidence 0.9 can indicate a high reliability while a confidence of 0.2 can indicate a low reliability. The confidence 608 can be used to modify the factor index 606, for example, by multiplying the factor index 606 by the confidence 608, and/or using any other algorithm for modifying the probability indicated by the factor index 606 as associated with a confidence 608. The confidence 608, the factor index 606 or both can be dynamically altered or modified based on user data and/or any other information.

The monitoring device 152 can determine an availability index 280 based on one or more correlating factors 602 associated with the user data received. The monitoring device 152 can use the one or more factor indexes 606, the one or more confidences 608, or both associated with the relevant correlating factor values 604 as an input to algorithm or a function, such as an averaging algorithm, to determine the availability index 280. For example, if the user data indicates that the time of day is early afternoon and that the activity level of the user is high, the monitoring device 152 can average the corresponding factor indexes (0.6+0.9)/2 so as to generate an availability index 280 of 0.75. If the availability index generated is at or exceeds a threshold, the monitoring device 152 and/or the service portal 150 can determine that the user is available for connection to a supporter or trusted user.

The service portal system 150 can receive the response from the monitoring device 152 at 510 and can send the response or one or more parts of the response (such as only the availability index 280) to the client device 4B as a user availability response at 512. The client device 4B can determine based on the response (for example, based at least on the user availability index 280) to initiate a connection with the monitoring device 152. At 514, the client device 4B can send a user connection request to the service portal system 150 which can send at 516 an initiate connection at 516 to the monitoring device 152. While FIG. 5 illustrates initiating a connection with the monitoring device 152, the present disclosure that the connection can be initiated between a client device 4B associated with a remote user and any network device associated with the user, such as any of a cellular telephone, a video conference system (such as any of a smart phone, a laptop, a tablet, any other I/O device, or any combination thereof), any other communication device, or any combination thereof. A connection is established at 518 such that the user and the remote user are able to communicate over the connection.

In one or more embodiments, the service portal system 150, the monitoring device 152 or both can record or otherwise store user data 270 associated with any of one or more successful connections, one or more unsuccessful connections, every type of connection, a selection of successful and/or unsuccessful connections, or any combination thereof as historical user data and can determine based on the historical user data to adjust one or more factors associated with the user data 270, such as any of alter one or more weights (for example, to adjust weights based on reality of successful connection versus predicted successful connection), such as one or more factor indexes 606, add and/or delete a type of sensing device 5 from which user data 270 can be received, alter a timing frequency, alter any other factor, or any combination thereof. In one or more embodiments, the one or more factors associated with the user data 270 can be adjusted dynamically, at predetermined intervals, based on a user input, any other period, or any combination thereof.

While FIG. 5 illustrates the client device 4B in communication with service portal system 150, the present disclosure contemplates that the communications 506-518 can be between the client device 4B and the monitoring device 152 directly and/or indirectly. In one or more embodiments, the monitoring device 152 is part of, within or otherwise connected in the cloud to the service portal system 150 such that one or more client devices 4 and/or one or more sensing devices 5 send user data to the monitoring device 152 via an access point device 2 as illustrated in FIG. 1. The present disclosure contemplates that any of the client device 4B, monitoring device 152, service portal system 150, or any combination thereof can be connected via one or more wired and/or wireless connections.

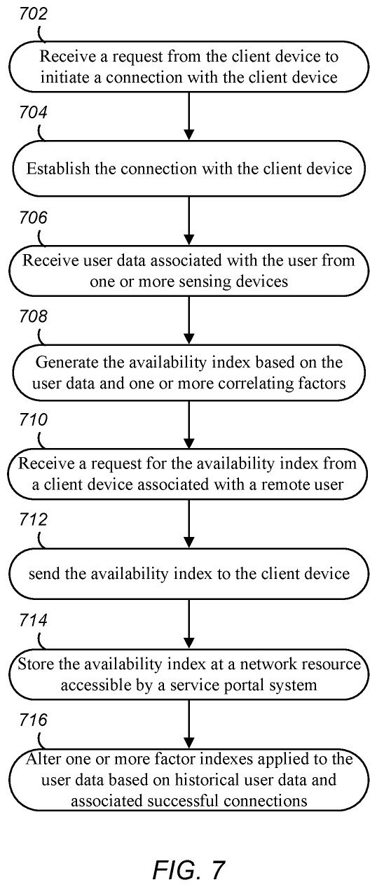

FIG. 7 is a flow chart illustrating a method for generating an availability index 280 associated with a user, according to one or more aspects of the present disclosure. For the steps of FIG. 7, reference to a network device 200 can comprise a service portal system 150, a monitoring device 152, including, but not limited to, a set-top box, a client device 4, or any combination thereof and may be programmed with one or more instructions that when executed by a processor controller causes the network device 200 to generate an availability index associated with a user, according to one or more embodiments. In FIG. 7, it is assumed that a network device 200 includes a respective controller and a respective software stored in a respective memory, as discussed herein in connection with any one or more figures which when executed by the respective controller performs the functions and operations in accordance with the example embodiments of the present disclosure (for example, including sending and receiving user data 270 and generating an availability index 280 associated with a user based on the user data 270).

At step 702, a network device 200 receives a request from a client device 4 associated with a remote user to initiate a connection with the client device 4. For example, a trusted user at a remote site, such as a medical professional at a health center, can request connection to the network device 200 so as to check on the availability of a user for a communication, for example, a medical examination or inquiry. At step 704, the network device 200 establishes a connection with the client device 4.

At step 706, the network device 200 receives user data associated with a user of the network device 200, for example, an aging-in-place elderly that subscribes to one or more services offered via the network device 200, such as monitoring of one or more sensing devices 5 associated with the user. The network device 200 can any of request, query, automatically receive, manually initiate sending of (for example, via a user intervention), any other inquiring, or any combination thereof the user data from the one or more sensing devices 5. For example, the one or more sensing devices 5 can at timed intervals send the user data to the network device 200 so as to not overburden or consume valuable resources associated with the network device 200 and/or the network.

At step 708, the network device can generate an availability index based on the user data and one or more correlating factors. For example, the user data received from the one or more sensing device 5 can be associated with one or more correlating factors, such as illustrated in FIG. 6. The network device 200 can determine that the user data is indicative of one or more corelating correlating factor values of the one or more correlating factors. The network device 200 can use one or more factor indexes associated with the one or more correlating factor values as an input to an availability algorithm or function, such as an averaging function. The network device can generate an availability index associated with the user. The network device 200 can any of routinely, automatically, manually, by command or request, or any combination thereof generate the availability index. For example, as indicated by step 710, the network device 200 can receive a request for the availability index from a client device associated with a remote user, such as a trusted user or a supporter associated with the user based on a profile configuration associated with the user.

At step 712, The network device 200 can send the availability index to the client device associated with the remote user. The network device 200 can be directly and/or indirectly, wired and/or wirelessly coupled to the client device 4. The network device 200 can store the availability index locally, remotely, for example, at a network resource assessable by a service portal system, or both.

At step 716, the network device 200 can alter one or more factor indexes applied to the user data based on historical user data and one or more associated successful connections, for example, both of which stored locally, remotely, or both. For example, the network device 200 can store historical user data along with whether that historical user data is associated with a successful connection between the trusted user and the user so that a factor index associated with the user data can be altered to more accurately reflect the availability of the user. As an example, the user data can indicate an activity level of high which can have an associated factor index of 0.9 as shown in FIG. 6. The associated successful connections based on this factor index, however, can indicate that the user is not always available when activity level of high is indicated by the user data. The network device 200 can alter the factor index associated with the correlating factor value of high for the correlating factor of activity level to 0.7 to more accurately reflect the availability of the user based on this factor index. In this way, the network device 200 can automatically maintain the one or more factor indexes to accurately reflect user availability.

Thus, according to one or more novel solutions of the present disclosure, a remote user can make an informed determination as to the availability of a user based on an availability index generated by a network device, such as a monitoring device, so as to conserve resources. Using the availability index allows a remote user to initiate a connection to the user at a time that the user is most likely available as indicated by the availability index so that the remote user does not waste time and incur costs associated with a failed connection attempt with the user. While FIG. 6 illustrates steps in a certain order, the present disclosure contemplates that any one or more steps of FIG. 6 can be implemented in any order or not at all (omitted).

While the present disclosure discusses an aging-in-place or healthcare environment generally, the present disclosure contemplates any other environment that requires establishing a remote connection between a remote user and a user so as to provide a service, such as any of a diagnostic service, caregiver service, any other service, aging-in-place service, or any combination thereof.

Each of the elements of the present invention may be configured by implementing dedicated hardware or a software program on a memory controlling a processor to perform the functions of any of the components or combinations thereof. Any of the components may be implemented as a CPU or other processor reading and executing a software program from a recording medium such as a hard disk or a semiconductor memory, for example. The processes disclosed above constitute examples of algorithms that can be affected by software, applications (apps, or mobile apps), or computer programs. The software, applications, computer programs or algorithms can be stored on a non-transitory computer-readable medium for instructing a computer, such as a processor in an electronic apparatus, to execute the methods or algorithms described herein and shown in the drawing figures. The software and computer programs, which can also be referred to as programs, applications, components, or code, include machine instructions for a programmable processor, and can be implemented in a high-level procedural language, an object-oriented programming language, a functional programming language, a logical programming language, or an assembly language or machine language.

The term “non-transitory computer-readable medium” refers to any computer program product, apparatus or device, such as a magnetic disk, optical disk, solid-state storage device (SSD), memory, and programmable logic devices (PLDs), used to provide machine instructions or data to a programmable data processor, including a computer-readable medium that receives machine instructions as a computer-readable signal. By way of example, a computer-readable medium can comprise DRAM, RAM, ROM, EEPROM, CD-ROM or other optical disk storage, magnetic disk storage or other magnetic storage devices, or any other medium that can be used to carry or store desired computer-readable program code in the form of instructions or data structures and that can be accessed by a general-purpose or special-purpose computer, or a general-purpose or special-purpose processor. Disk or disc, as used herein, includes compact disc (CD), laser disc, optical disc, digital versatile disc (DVD), floppy disk and Blu-ray disc. Combinations of the above are also included within the scope of computer-readable media.

The word “comprise” or a derivative thereof, when used in a claim, is used in a nonexclusive sense that is not intended to exclude the presence of other elements or steps in a claimed structure or method. As used in the description herein and throughout the claims that follow, “a”, “an”, and “the” includes plural references unless the context clearly dictates otherwise. Also, as used in the description herein and throughout the claims that follow, the meaning of “in” includes “in” and “on” unless the context clearly dictates otherwise. Use of the phrases “capable of,” “configured to,” or “operable to” in one or more embodiments refers to some apparatus, logic, hardware, and/or element designed in such a way to enable use thereof in a specified manner.

While the principles of the inventive concepts have been described above in connection with specific devices, apparatuses, systems, algorithms, programs and/or methods, it is to be clearly understood that this description is made only by way of example and not as limitation. The above description illustrates various example embodiments along with examples of how aspects of particular embodiments may be implemented and are presented to illustrate the flexibility and advantages of particular embodiments as defined by the following claims, and should not be deemed to be the only embodiments. One of ordinary skill in the art will appreciate that based on the above disclosure and the following claims, other arrangements, embodiments, implementations and equivalents may be employed without departing from the scope hereof as defined by the claims. It is contemplated that the implementation of the components and functions of the present disclosure can be done with any newly arising technology that may replace any of the above-implemented technologies. Accordingly, the specification and figures are to be regarded in an illustrative rather than a restrictive sense, and all such modifications are intended to be included within the scope of the present invention. The benefits, advantages, solutions to problems, and any element(s) that may cause any benefit, advantage, or solution to occur or become more pronounced are not to be construed as a critical, required, or essential features or elements of any or all the claims. The invention is defined solely by the appended claims including any amendments made during the pendency of this application and all equivalents of those claims as issued.

What we claim is:

1. A network device for generating an availability index associated with a user, comprising:
a memory storing one or more computer-readable instructions; and
a processor configured to execute the one or more computer-readable instructions to:
receive user data associated with the user from one or more sensing devices;
generate the availability index based on the user data and one or more correlating factors;
receive a request for the availability index from a client device associated with a remote user; and
send the availability index to the client device.

2. The network device of claim 1, wherein the processor is further configured to execute the one or more instructions to:
apply a factor index associated with the one or more correlating factors to the user data, wherein the availability index is generated based on applying the factor index to the user data.

3. The network device of claim 1, wherein the processor is further configured to execute the one or more instructions to at least one of:
receive a request from the client device to initiate a connection with the client device; and
establish the connection with the client device.

4. The network device of claim 1, wherein the processor is further configured to execute the one or more instructions to:
store the availability index at a network resource accessible by a service portal system.

5. The network device of claim 1, wherein the availability index is based on a profile configuration associated with the user.

6. The network device of claim 1, wherein the network device is a monitoring device that comprises at least one of the one or more sensing devices.

7. The network device of claim 1, wherein the processor is further configured to execute one or more instructions to:
alter one or more factor indexes applied to the user data based on historical user data and one or more associated successful connections.

8. A method for generating an availability index associated with a user, the method comprising:
receiving user data associated with the user from one or more sensing devices;
generating the availability index based on the user data and one or more correlating factors;
generating the availability index based on the user data and one or more correlating factors; and
sending the availability index to the client device.

9. The method of claim 8, further comprising:
applying a factor index associated with the one or more correlating factors to the user data, wherein the availability index is generated based on applying the factor index to the user data.

10. The method of claim 8, further comprising:
receiving a request from the client device to initiate a connection with the client device; and
establishing the connection with the client device.

11. The method of claim 8, further comprising:
storing the availability index at a network resource accessible by a service portal system.

12. The method of claim 8, wherein the availability index is based on a profile configuration associated with the user.

13. The method of claim 8, wherein the network device is a monitoring device that comprises at least one of the one or more sensing devices.

14. The method of claim 8, further comprising:
altering one or more factor indexes applied to the user data based on historical user data and one or more associated successful connections.

15. A non-transitory computer-readable medium of a network device storing one or more instructions for generating an availability index associated with a user of the network device, which when executed by a processor of the network device, cause the network device to perform one or more operations comprising:
receiving user data associated with the user from one or more sensing devices;
generating the availability index based on the user data and one or more correlating factors;
receiving a request for the availability index from a client device associated with a remote user; and
sending the availability index to the client device.

16. The non-transitory computer-readable medium of claim 15, wherein the one or more instructions when executed by the processor further cause the network device to further perform the one or more operations comprising:
applying a factor index associated with the one or more correlating factors to the user data, wherein the availability index is generated based on applying the factor index to the user data.

17. The non-transitory computer-readable medium of claim 16, wherein the one or more instructions when executed by the processor further cause the network device to further perform the one or more operations comprising:
receiving a request from the client device to initiate a connection with the client device; and
establishing the connection with the client device.

18. The non-transitory computer-readable medium of claim 8, wherein the one or more instructions when executed by the processor further cause the network device to further perform the one or more operations comprising:
storing the availability index at a network resource accessible by a service portal system.

19. The non-transitory computer-readable medium of claim 15, wherein at least one of:
the availability index is based on a profile configuration associated with the user; and
the network device is a monitoring device that comprises at least one of the one or more sensing devices.

20. The non-transitory computer-readable medium of claim 15, wherein the one or more instructions when executed by the processor further cause the network device to further perform one or more operations comprising:
altering one or more factor indexes applied to the user data based on historical user data and one or more associated successful connections.
